(19) United States
(12) Patent Application Publication
(10) Pub. No.: US 2008/0262068 A1
(43) Pub. Date: Oct. 23, 2008

(54) Method of Tonic Treatment With Oxyphenbutazone Derivatives

(75) Inventor: Jørgen Karlsen, Lysaker (NO)

Correspondence Address: BACON & THOMAS, PLLC, 625 SLATERS LANE, FOURTH FLOOR, ALEXANDRIA, VA, 22314-1176, US

(73) Assignee: A-Viral ASA, Lysaker (NO)

(21) Appl. No.: 11/596,278

(22) PCT Filed: May 10, 2005
(86) PCT No.: PCT/GB05/01772

§ 371 Date: Nov. 13, 2006

(30) Foreign Application Priority Data
May 12, 2004 (NO) 20041947

(51) Int. Cl. A61K 31/4152; A61P 43/00

(52) U.S. Cl. 514/404

(57) ABSTRACT

The present invention provides a method of tonic treatment of an aging mammalian subject, or a subject suffering from mild inflammation, lupus, fatigue, lethargy or the aftereffects of infection, disease or treatment, comprising administration of a compound of formula (I) or a salt thereof. Said compounds are also used as a support to another drug and/or treatment regime especially a method for the treatment of a viral, autoimmune, hyperplastic or neoplastic disease, and for the prevention of diseases associated with old age including arthritis (especially RA), Alzheimer's, Parkinson's and Huntington's diseases, cancer or other neoplastic disease.

The present invention relates to a method of tonic treatment of a subject by administration of certain substituted 4-hydroxyoxyphenbutazone compounds. More particularly, the present invention relates to such treatment in the amelioration of the symptoms of lethargy, allergy, fatigue or old age.

The cyclic pyrazolidine dione compounds phenbutazone, oxyphenbutazone and 4-hydroxy oxyphenbutazone are known or suggested in the treatment of inflammatory, viral and/or autoimmune diseases.

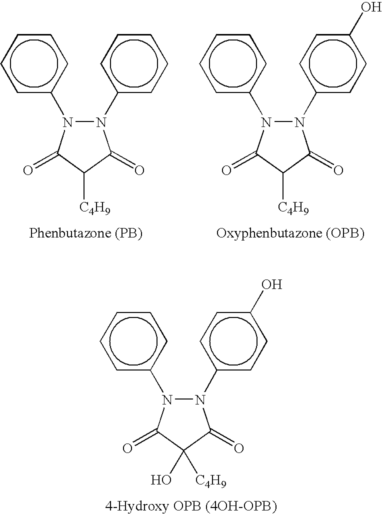

Many variants of these pyrazole based structures have been investigated, including derivatives (e.g. U.S. Pat. No. 3,968,219), and prodrugs (e.g. U.S. Pat. No. 4,117,232, U.S. Pat. No. 3,957,803, U.S. Pat. No. 4,169,147, U.S. Pat. No. 4,036,845 and U.S. Pat. No. 4,139,709). The principal work on those with biological activity has, however, related to varying the makeup of and substituents on the central pyrazolidine core.

The present inventors have now, unexpectedly, established that compounds of the 4OH—OPB class, may be used in “tonic” treatment, especially when used at dosages lower than previously known.

In a first aspect, the present invention therefore provides a method of tonic treatment of an aging mammalian (preferably human) subject, or a subject suffering from mild inflammation, allergy, lupus (especially cutaneous lupus), fatigue, lethargy or the aftereffects of infection, disease or treatment, comprising administration of a compound of formula I or a salt thereof;

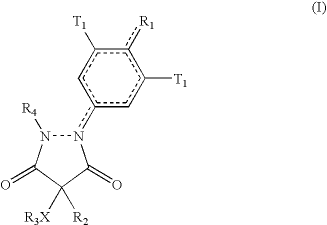

wherein R₁ is H, OH, SH, O-alkyl, S-alkyl, O-acyl or S-acyl, in which case the bond N— —N exists and the six-membered ring is aromatic, or R₁ is O or S joined by a double bond, in which case the bond N— —N is absent and the six-membered ring is 2,5-unsaturated; R₂ is hydrogen or more preferably an C₁-C₁₀ organic group attached by a carbon atom, e.g. an optionally substituted alkyl, alkenyl, alkynyl, aryl, alkaryl, aralkyl, aralkenyl or arylsulphonylalkyl group; X is H, O, OO, S or SS; R₃ is absent (where X=H), is hydrogen or is a hydroxyl or thiol protecting group (e.g. a (preferably C₂-C₇) acyl, or alkaryl group, such as an acetyl or benzyl group) R_{d} is a hetero- or preferably homo-cyclic aryl group, optionally substituted with a further group R₅ (e.g. with an alkyl, alkenyl or alkynyl group, OH, O-alkyl, thio, thioalkyl, halo, or primary, secondary, tertiary or quaternary amino group); and groups T₁ are each, independently, hydrogen or an S—R₆ group, where each R₆ is independently an organic group of molecular weight up to around 500 amu, such as a substituted or unsubstituted alkyl, alkenyl, alkynyl, alkaryl, aralkyl, alkyl ester, alkyl amide, alkyl acid, polyol, sugar, oligo(alkylamide), oligo(alkylester), or oligopeptide group.

Such compounds are phenbutazones, oxyphenbutazones or 4-hydroxyoxyphenbutazones, or open-chain equivalents thereof and are commercially available or synthesised by methods described herein and by methods known in the art, such as from WO 01/00585 and the references cited therein. The disclosure contained in this document and in all references cited herein is hereby incorporated herein by reference.

Specifically, 4-hydroxyoxyphenbutazones may be synthesised from oxyphenbutazones by oxidation of corresponding compounds in which the R₃X position is occupied by hydrogen; from other 4-OH OPBs by reaction of corresponding compounds in which R₃X is HX with hydroxy or thiol protecting groups to introduce non-hydrogen R₃ group, or by condensation of a hydrazine compound with an optionally protected 2-hydroxy-propane dioic acid halide, ester or similar compound, e.g.

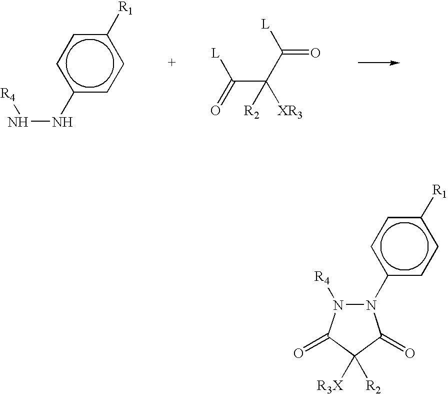

wherein R₁-R₄ and X are as defined above or protected equivalents or precursors thereof and the groups L are leaving groups such as halides etc. Where X is H, oxyphenbutazones will result, which may be converted to 4-OH OPBs as described above. Where X is O, 4-OH OPBs will be formed directly.

As will be readily appreciated, the hydrazines may be prepared by hydrogenation of the corresponding diarylazo compounds (since R₄ is aryl), which in turn can be synthesised from simple aromatic nitro compounds in the presence of LiAlH₄.

The open-chain compounds (wherein R₁ is joined by a double bond, the six-membered ring is 2,5 unsaturated and bond N— —N is absent) may be synthesised simply by ring-opening of the (optionally protected) equivalent cyclic compound. This can be carried out under mild conditions and is illustrated in the Examples below.

The present inventors have, unexpectedly, established that compounds of the present invention have considerable utility as modulators the immune system within the body and provide a “tonic” effect in subjects suffering from fatigue, allergy, inflammation, lethargy or the effects of aging, whether or not any direct, identifiable, cause of these symptoms is evident.

In a further aspect, the present invention provides the use of a compound of formula I or a salt thereof as defined herein in the manufacture of a medicament for the treatment of the symptoms of inflammation, fatigue, allergy, lethargy or old age or the aftereffects of infection, disease or treatment. Preferably, the compound will be a preferred compound as described herein.

Compounds of formula I or a salt thereof may be usefully administered in the form of a pharmaceutical composition, particularly for the treatment of disease. Alternatively, the compounds may be taken in the form of an “functional food”, a supplement or as a food or beverage fortification, particularly where a “tonic” effect in the reduction of the symptoms of fatigue, allergy, lethargy or old age or a general boost to the immune system is desirable.

In a yet still further aspect, the present invention therefore provides a pharmaceutical composition comprising a compound of formula I or a salt thereof as defined herein and at least one pharmaceutically acceptable excipient, carrier or diluent. The invention also provides a functional or fortified food comprising a compound of formula I or a salt thereof formulated in an edible food or beverage.

Preferred compounds of the invention are of formulae II and III, and salts thereof;

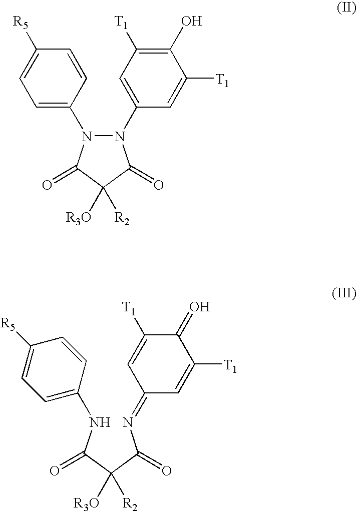

wherein T₁, R₂ and R₃ are as described above and R₅ is an alkyl, alkenyl or alkynyl group (such as those listed infra for R₂), an OH, O-alkyl, O-acyl, SH, S-alkyl, S-acyl, halo, or primary, secondary, tertiary or quaternary amino group. Preferred R₅ groups are hydrogen, OH and O-acyl (e.g O-acetyl). Most preferred are hydrogen, OH and O-acetyl.

In the compounds and starting materials of the invention, R₂ is preferably an arylsulphonylalkyl or C₁ to C₆ alkyl, alkenyl, or alkynyl group, e.g. a methylsulphonylphenyl, ethylsulphonylphenyl, methyl, ethyl, ethylenyl, acetylenyl, n-propyl, i-propyl, prop-1-enyl, prop-2-enyl, n-butyl, i-butyl, s-butyl, t-butyl, but-1-enyl, but-2-enyl, but-3-enyl, 1-methyl-prop-1-enyl, 1-methyl-prop-2-enyl, 2-methyl-prop-1-enyl, 2-methyl-prop-2-enyl, n-pentyl, i-pentyl etc. More preferably R₂ is C₂ to C₆ alkyl, particularly n-butyl, i-butyl, s-butyl or t-butyl. The most preferred R₂ group is n-butyl.

R₃ in the compounds and starting materials described herein is preferably hydrogen or a metabolically labile protecting group which yields a physiologically tolerable byproduct. Suitable protecting groups are acyl groups, particularly acetyl, propanoyl, methylpropanoyl or n-butanoyl. Many additional OH and SH protecting groups are however known (see e.g. Greene, “protective groups in organic synthesis”, Wiley Interscience, NY, 1981) and these may be of value as products, or particularly as intermediates. Most preferred R₃ groups are hydrogen and acetyl.

In the compounds and starting materials of the present invention, T₁ is preferably H or a thiol group substituted with an R₆ group (i.e. S—R₆), where R₆ is a targeting moiety or a small (esp MW<500) organic group having at least two functional groups selected from esters, amides, carboxylic acids, hydroxyl groups and amines. It is preferred that either both T₁ groups are H or both are thiols (although the R₆ groups of such thiols may be the same or different). Preferably, R₆ is an oligo ester or oligo peptide with at least one free acid and/or amine group. Examples of such groups include specific binding peptides such as antibody fragments. More preferably, each T₁ is independently hydrogen or a 1-5 residue oligo peptide. Most preferably, each T₁ is independently hydrogen or glutathione. That is to say, both T1 groups may be hydrogen, both may be glutathione, or one may be glutathione and the other hydrogen.

The most preferred compounds of the present invention are of formulae IV or V or salts thereof;

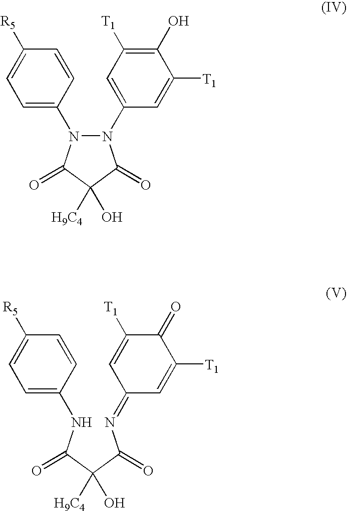

wherein R₅ is hydrogen or OH and each T₁ is independently H or glutathione.

Compounds of the present invention may be formulated as pharmaceuticals by methods well known in the art. These formulations will typically be oral formulation such as tablets, coated tablets (such as controlled release tablets), capsules, suspensions, solutions, syrups, powders, or emulsions but may be formulations for inhalation (such as powders or aerosols), transdermal absorption (such as patches) or for parenteral (e.g subcutaneous, intramuscular or intravenous) ocular or rectal administration in the form of, for example, sterile saline solutions, drops or suppositories.

The compounds of formula I and salts thereof may be formulated with conventional pharmaceutical carriers, diluents and/or excipients such as aqueous carriers (e.g. water for injections), binders, fillers, stabilizers, osmolality adjusting agents, effervescing agents, pH buffers and modifiers, viscosity modifiers, sweeteners, lubricants, emulsifiers, flavours, coating agents (e.g. gastric juice resistant coatings) etc.

The dosage of the compounds of formula I or salts thereof administered to a subject will be dependent upon the species, size, maturity, health and condition of the subject and upon the formulation chosen. Inhalable or intravenous formulations, for example, may deliver a larger proportion of the active agent to the subject than oral formulations but in general oral formulations will be preferred. Generally, doses will be in the range of 1 to 2000 mg/day, more typically 2 to 1000 mg/day, especially 5 to 500 mg/day. Administration will typically be once, twice, three or four times per day but may more or less often (e.g. five or six times per day, once every two or three days, or every time symptoms are detected) if appropriate.

The compounds of formula I may be administered as a tonic, such as to reduce lethargy, mild (especially chronic) inflammation, as a treatment or especially a prophylaxis in allergy, to combat the symptoms of old age or to boost the immune system and they may be formulated as pharmaceuticals as above. Alternatively, the compounds may be formulated as functional foods or beverages, in which situation the carriers and excipients will typically comprise edible food or beverage products. Such products may be processed foods for consumption hot, such as ready meals, but will more preferably be cold foods including spreads (e.g margarine or low-fat spreads), jams, still or carbonated soft drinks, fruit juice, breakfast cereals, breakfast bars, breads, biscuits, ice-creams, chilled desserts such as yoghurts, mousses or trifles, milk or milk based drinks and the like.

Where the compounds of formula I are formulated as functional foods or beverages, it will be important that the maximum dose which can be accidentally consumed by over-eating such foods is not excessive. In such cases, the dosage present in one portion of such functional foods will typically be no more than 1 000 times less than the lethal dose, more preferably no more than 10 000 times less and most preferably no more than 50 000 times less than the human lethal dose.

Where the compounds of the invention are referred to herein as salts, these will generally be pharmaceutically acceptable salts i.e. those with physiologically tolerable counterions. Such ions include sodium, calcium, organic amines, halides (especially chloride), phosphates, hydrogen carbonates etc.

Without being bound by theory, the effect of the compounds, and compositions of the invention is believed in part to be the result of stimulating and modulating a “cleanup” effect, in which the body is stimulated to remove not only infectious agents but also cell debris and other unwanted matter. In addition, the compounds of the invention may show effects as T-lymphocyte and monocyte activation inhibitors and modulators of interleukins.

By such a processes, the tendency for biological debris to accumulate is reduced and the immune system refocused. As a result, the subject is provided with a better quality of life and the body purged of some unnecessary and even detrimental waste. This tonic effect may be applied during or following treatment for a primary disease, condition or infection, or may be an end in itself, when, for example, infection, drug treatment, chronic inflammation or the aging process has resulted in compromised function or a build up of unwanted matter in the system.

The modulation of Acute Phase Proteins (APP) in particular is believed to induce a cleanup of the system, removing cell debris. The breakdown products of host cells can induce the death of neighbouring cells, thereby causing a cascade of cell death and thus their removal supports the wellbeing of healthy tissue. Unusually, the compounds of the present invention may stimulate acute phase proteins without inducing significant fever and are not typically general immune-suppressants.

The compounds of the present invention have been shown to modulate the production of interleukin-6, which in turn affects acute phase protein production. In particular, at low to moderate dosage levels, the compounds of the present invention have been observed to stimulate interleukin production by up to 50%, but higher doses can essentially eliminate production of the same interleukins. It is also noticeable that the compounds of the present invention modulate monokine production and production of Th1 and Th2 specific cytokines. These types of cells, particularly monocytes and Th2 cells are known to contribute to inflammatory reactions and thus effects on such cells and their cytokines may contribute to allowing the immune system to be stimulated without inducing inflammation. The dosage level required to provide the desired immuno-modulatory effect can be established for any particular situation by standard methods and particularly by reference to the Examples herein. In general, a moderate dose of, for example, up to 1000 mg, preferably up to 500 mg and more preferably up to 350 mg in a single application will provide a boost immune function and cytokine production. Correspondingly, if a multiple-dose regime is adopted the amounts will be correspondingly smaller. The minimum valuable dose for such effects may be as little as 1 mg per dose, since this may be formulated in, for example, a food product where several doses can be taken together or successively. This minimum amount will more generally be at least 5 mg, preferably at least 10 mg and more preferably at least 20 mg, subject to the toxicity constraints indicated herein. One measure of the effective dose which can readily be established by skilled workers is the peak concentration in the bloodstream of a patient. This concentration will relate directly to the effectiveness of the agent irrespective of the dosage regime used and may be measured by taking samples of whole blood at times after administration to a test sample of patients. In one aspect, the preferred dose in the present invention achieves a moderate maximum bloodstream concentration. Such a concentration may be, for example, 0.3 to 160 μM, preferably 0.5 to 100 μM, more preferably 0.5 to 80 μM and most preferably 1 to 40 μM.

In one particularly preferred aspect of the present invention, the compounds of the invention are administered at the low to moderate dosage level described above, such that the bloodstream concentration achieves a level in the region of stimulation of immune processes. These concentrations include those indicated in the previous paragraph. If the bloodstream concentration is allowed to rise too high then these same immune processes can be inhibited, as indicted in the Examples below. It is thus a crucial and unexpected factor of this aspect of the invention that selection of the correct dosage can provide the desired effect while higher dosages provide an opposing effect. In particular, appropriate low to moderate doses can preferentially stimulate the non-specific parts of the immune system, e.g. to carry out “cleanup” processes as described herein. Without being bound by theory, it is believed that it is this stimulation of the non-specific immune system which is the common factor allowing appropriate dosages of the compounds described herein to provide advantageous effects in conditions including old age, lethargy, allergy, and inflammation. In all cases the stimulation of the appropriate part of the immune system is important and in some cases it is critical that this be the non-specific system rather than more specifically directed (e.g. t-cell mediated) responses.

An alternative method for bringing about a cleanup of biological debris is binding by certain plasma proteins such as particular immunoglobulin Ms (IgMs) with specificity for the membrane phospholipids of dead (but not living) cells, b2 glycoprotein I, clusterin and serum amyloid P. Such mechanisms may also be modulated by the compounds of the present invention.

The tonic effect of the compounds of the present invention in older subjects may also be explicable as a result of a cleanup mechanism. As subjects age, a greater proportion of cells suffer programmed cell death due to telomere reduction and apoptosis. At the same time, the level of clean-up mechanisms such as APPs and the effectiveness of the immune system typically declines. This may lead to a build up of debris, chronic inflammation and a susceptibility of infection. These factors may then ultimately lead to degenerative diseases and conditions such as heart attacks or strokes. By prophylactic treatment with the compounds of the present invention, the debris buildup may be reduced, freeing the immune system to rid the body of infections before catastrophic events such as bursting of blood vessels causes conditions such as heart attacks. Modulation of inflammation may also contribute to avoidance of such problems.

Similarly, a build up of biological debris is a particular problem in diseases associated with old age such as arthritis, Alzheimer's, Parkinson's and Huntington's diseases. The compounds of the invention may thus be valuable both in treating and in preventing the onset or worsening of such conditions.

In a preferred embodiment, the invention also provides a method of prophylaxis against the development of diseases associated with old age including arthritis (especially RA), Alzheimer's, Parkinson's and Huntington's diseases, cancer or other neoplastic disease, the method comprising administration of a compound of formula I, or a salt thereof.

In a similar way to that seen in aging subjects, those suffering or from chronic disease may experience a build up of biological debris from both host cells and infectious agents and a tendency to chronic inflammation. The compounds of the present invention may be administered to speed recovery and improve quality of life in such cases. This mechanism is also suitable for speeding the recovery of any subject after events such as malaria, surgery, burns or sepsis.

With regard to the treatment, and/or prophylaxis of allergic conditions, the compounds of the present invention can provide both reactive and particularly pre-emptive effects. Specifically, and again without wishing to be bound by theory, by enhancing the response of the non-specific parts of the immune system, such as macrophages, the compounds of the present invention may cause potentially allergenic substances to be cleared from the body quickly by this route. Clearance in this way provides less activation of the specific immune system and less chance of providing a comprehensive, specific immune response. This therefore decreases the inflammation caused and avoids “priming” the immune system against the allergen. In contrast, if an allergen is not cleared rapidly then there is a greater chance of the manufacture of specific binders to the allergen, e.g. by t-cells, which in turn would increase the strength of the allergic reaction on the next exposure.

In one aspect of the invention, the compounds and compositions of the present invention are thus used in the treatment and/or prophylaxis of, or manufacture of a medicament for the treatment and/or prophylaxis of, at least on allergic condition. Suitable examples include pollen/airborne particle allergies (e.g. hay fever), food allergies (e.g. nut allergies, seafood allergies etc.), drug allergies (e.g. to aspirin or β-lactam antibiotics), and skin allergies (e.g. to latex, sticking plaster, chemical initiators etc).

The effects of the compounds of the invention may advantageously be used in combination with other drugs, particularly to improve the quality of life of subjects suffering symptoms of a primary condition or side-effects from the treatment therefor. For example, subjects suffering from a hyperplastic or neoplastic disease such as cancer or leukemia may be treated with one or more cytotoxic agents (such as nucleoside analogues), by surgery, external beam irradiation and/or radionuclide therapy. Patients having undergone transplants or with certain autoimmune diseases may be treated with powerful immune-suppressants leaving them vulnerable to infection in a similar way to patients with immune systems compromised directly by the effects of diseases such as HIV infection. In addition, the compounds or compositions of the present invention may be administered to free up and focus the immune response (particularly, for example by the stimulation of macrophages) against any remaining tumour cells, micro-tumours or micro-metastases in order to provide more complete remission of the disease. Such treatment may be carried out during or after treatment by other agents or interventions.

The compounds of the present invention may also be used to stimulate the destruction particularly by macrophages) of micro-tumours and thereby prevent the formation or spread of neoplastic disease. This may be employed prophylactically, particularly in older subjects (see below) or those considered as having a predisposition to neoplastic disease (e.g. due to heredity; exposure to predisposing drugs or chemical or physical environments, such as certain hormones, carcinogens, ionising radiation, etc; previous treatment for neoplastic disease; results of genetic testing etc).

An additional benefit of the effects of the compounds of the present invention is in combination with other immune modulators and in particular in combination with powerful immune suppressants and cytokine production inhibitors. In cases such as autoimmune disease, where a powerful immune suppressant is administered, the prior, simultaneous and/or subsequent administration of compounds of the present invention may restore certain immune function without disrupting the desirable function of the primary medication. In this way the patient achieves a better quality of life by having a lower susceptibility to infection than would otherwise occur. Examples of conditions suitable for treatment in this way include autoimmune diseases such as Rheumatoid Arthritis (RA) or systemic lupus.

In a further preferred aspect, the present invention therefore provides a method for the treatment of a mammalian (preferably human) subject comprising administration of a compound of formula I or a salt thereof as defined herein, as a support to another drug and/or treatment regime. Preferably, the method is a method for the treatment of a viral, autoimmune, hyperplastic or neoplastic disease, more preferably for the treatment of HIV, RA, lupus, cancer or leukaemia. The other drug is preferably an antiviral, such as those listed herein, an immune suppressant, or an antineoplastic agent such as a radiopharmaceutical or chemotherapeutic (e.g. asparaginase, bleomycin, cisplatin, cladribine, cyclophosphomide, cytrabine, dacarbazine, daunorubicin, doxorubicin, etoposide, fluorouracil, hydroxyurea, mercaptopurine, mustine, methotrexate, procarbazine, or vinblastine). The other treatment regime is preferably surgery and/or external beam irradiation. In this method, the compound of the present invention will typically be formulated as a pharmaceutical, administered prior to or preferably concurrently with or after the other drug or treatment.

Where symptoms such as fatigue or lethargy are the result of old age or viral, bacterial or fungal infection or result from the symptoms or treatment of hyperplastic disease such as cancer, the compounds of the present invention may be administered either as a pharmaceutical, or as an additive in, for example a “functional food”. One particular advantage of a functional food is that a subject can easily establish a routine of consuming small doses several times per day and so maintain the important low, but therapeutic, concentration of active agent, as described herein.

Where the cause is a medical condition or treatment, the compound of the invention will generally be taken in the form of a pharmaceutical. Where, however, the cause is simply the result of the general build up of unwanted debris in old age or as the result of chronic, low intensity, inflammation for which no specific treatment is prescribed, the compounds of the present invention will preferably be taken in the form of a functional food or dietary supplement for convenience and ease of compliance.

In a the method of tonic treatment of an aging mammalian (preferably human) subject, this subject will generally be in the last quarter of the average life-span of such a mammal, more preferably the last 10% of such an average life-span and may be as old or older than this average. Where the subject is an aging human they will preferably be at least 60 years of age, more preferably at least 70 and most preferably at least 75. The subject may be suffering from an identifiable viral, inflammatory, immune-deficient, autoimmune or allergic disease or condition, or may be a generally healthy subject in these or all respects wishing for a boost in physical or mental energy or in immune function or a reduction in fatigue or lethargy. The invention also provides for the use of the compounds of the invention in the manufacture of a tonic medicament suitable for use in such methods.

In one preferred aspect, the present invention relates to tonic treatment in patients with no specific or identified viral disease. In a further preferred aspect, the invention relates to tonic treatment in patients with no specific or identified inflammatory or autoimmune disease, in particular no acute inflammatory or autoimmune disease. In this aspect, the invention relates rather to prophylaxis against such disease and/or treatment of low-grade chronic or intermittent inflammatory or autoimmune disease with symptoms sufficiently mild or general as to not indicate the use of established treatments.

The invention also relates to the prophylactic treatment of patients susceptible to allergic reactions, in order to prevent or reduce the generation of allergies and/or further allergies. For example, such subjects may be allergic to some or many stimuli and may be treated in order to prevent the debilitating development of wide-scale allergies. Alternatively, such patients may have a suspected predisposition to allergies (e.g. by inheritance, due to exposure to allergens etc or discovered by screening, such as genetic screening) and may be treated prior to the onset of any significant allergic response.

The present invention will now be illustrated by the following, non-limiting examples.

### EXAMPLES

¹H-NMR were recorded on a Bruker 300 MHz spectrometer with CDCl₃ as solvent. HPLC was performed with a Gynkotek pump equipped with a Symmetry C-18, 5 mm, 3.9×150 mm column and a Gynkotek UVD 170S detector set at 254 nm. Gradient: 1% TFA in water/acetonitrile 70/30 to 0/100 in 8 min.

### Example 1

### Synthesis of 4OH—OPB

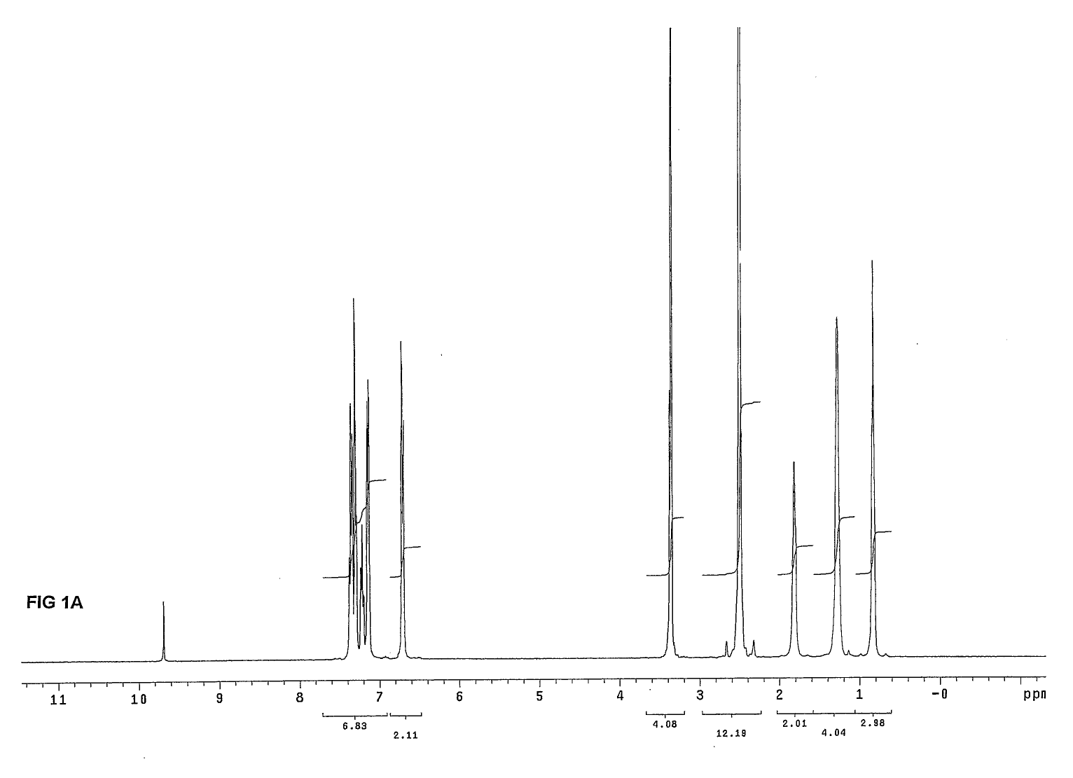

To a 1-litre round bottom flask with magnetic stirring is charged methanol (450 ml) and oxyphenbutazone hydrate (90.0 g, 0.26 mol). The solution is stirred at ambient temperature and sodium hydroxide solution (2M, 13.5 ml) is added. Hydrogen peroxide (30%, 180 ml) is added drop wise over 10 min. The resulting clear pale yellow solution was stirred for 24 h. The resulting suspension was cooled on an ice bath for 2.5 h and the mixture filtered through a glass filter and sucked dry. The light brown crystals were washed carefully on the filter with MeOH/water (1:2, 200 ml), sucked dry and washed once more with 100 ml of the same solvent mixture. The product was allowed to dry on the filter over night. The crude product was then transferred to a 200 ml round bottom flask, diethyl ether (200 ml) added and the resulting suspension stirred vigorously for approximately 5 min. The mixture was filtered and sucked dry on the filter. The appearance of the product was pale pink after the ether treatment. Crude yield 53 g. The ether treatment procedure was repeated once more with 150 ml of ether. The now almost white material was dissolved in methanol (330 ml) to give a red solution. Water (350 ml) was charged slowly over 35 min to give a white suspension. The solid was collected on a glass filter and dried in vacuo at 30° C. over night to give 4-OH OPB as a pale pink solid, 31 g, 35%. HPLC>98%. ¹H NMR confirms identity with reference sample. FIG. 1A shows the full ¹H NMR spectrum. FIG. 1B shows and aromatic region expansion.

### Example 2

### Thiol Derivitisation

The 4OH—OPB of Example 1 was derivatised with glutathione by incubation in glutathione (GSH) solution followed by purification. Conditions were chosen such that approximately equal quantities of the mono-glutathione substituted product (4OH—OPB-1GSH) and di-glutathione substituted product (4OH—OPB-2GSH) resulted.

### Incubation

4OH—OPB (170 mg) was dissolved in PBS (100 ml, formulated as below) additionally containing 1.5 mM glutathione. The solution was incubated for 30 minutes at 371 C and the reaction followed by Mass Spectrometric analysis.

### Purification

An analytical HPLC run (C18 reversed phase column) was performed to validate the products formed (determination by Mass Spectrometry). The 4OH—OPB-1 GSH and 4OH—OPB-2GSH were then purified by loading all 100 mL of the incubation mixture on a preparative column (C18, reversed phase column).

Both analytical and preparative runs were eluted with gradient eluents, running from 0% acetonitrile to 67% acetonitrile (in deionised water) in the presence of 0.1% TFA to keep the pH at 2. During the preparative run, fractions were collected (peaks) and checked for the right product by Mass Spectrometry. Finally, the identified products were dried under vacuum leaving products with ˜99% purity (as judged by MS).

PBS=(phosphate buffered saline, pH 7.5)

NaCl, 8.2 g; Na₂BPO₄.2H₂O, 1.9 g; NaH₂PO₄.H₂O, 0.3 g; Na+, 163, 9 mM; Cl⁻, 140, 3 mM; HPO₄²⁻, 10, 9 mM; H₂PO⁴⁻, 1, 8 mM, Braun Melsungen AG.

### Example 3

### Suppression of Cytokine Production

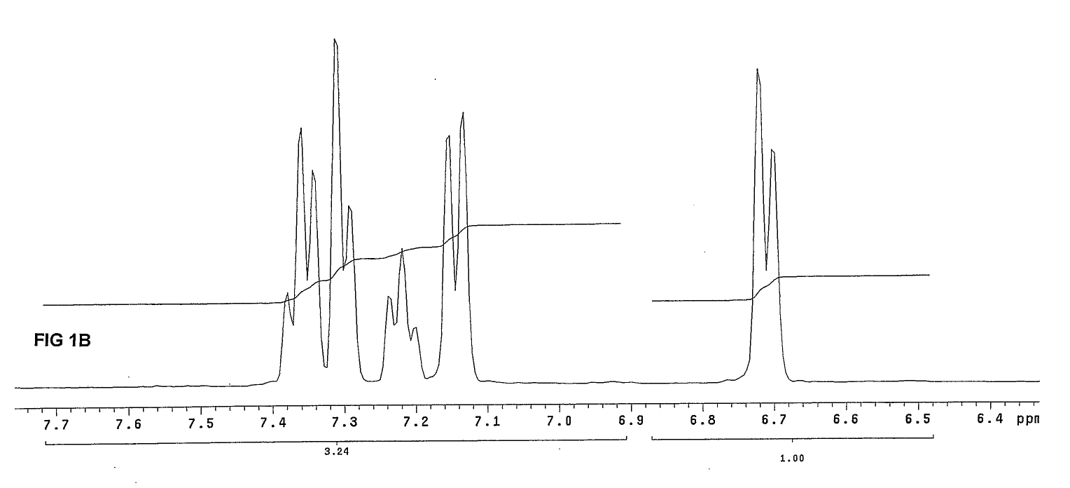

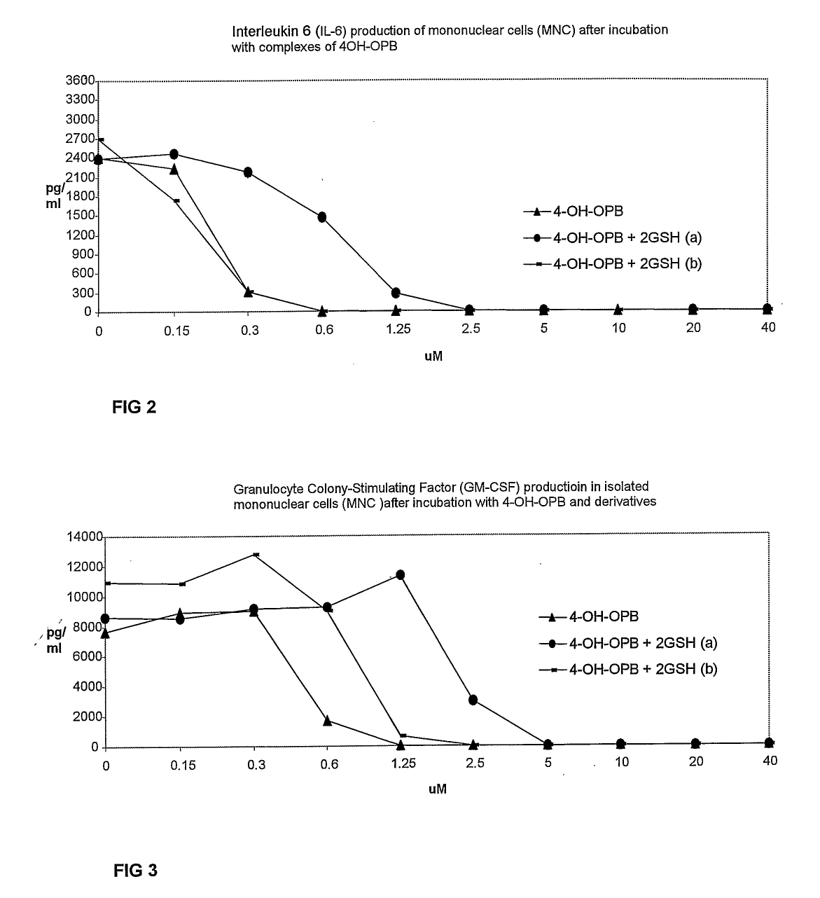

The 4OH—OPB as prepared in Example 1 and two batches of the 4OH—OPB-2GSH, as prepared in Example 2, were incubated with isolated human mononuclear cells (MNC) derived from peripheral blood from healthy volunteers. Production of the cytokines Interleukin-6 (IL6) and Granulocyte Colony-Stimulating Factor (GM-CSF) was measured. The results (shown in FIGS. 2 and 3) indicate that 0.5-5 FM of either product is sufficient to completely block production of the measured cytokines.

### Example 4

### Ring Opening

Conversion 4-Hydroxy-oxyphenbutazone (4-OH—OPB) into N-phenyl-2-hydroxy-2-butyl-1,3-propanal quinoneimine (PHBPQ) was carried out by incubation in buffer at pH 7.4, followed by purification by High Performance Liquid Chromatography (HPLC).

### Incubation

4-OH—OPB (34 mg) was first dissolved in DMSO (1 ml) before incubation for 15 minutes at 37° C. in PBS to a concentration of 100 FM (100 ml, PBS constituted as below).

### Purification

An analytical HPLC run (C18 reversed phase column) was performed to validate the products formed (determination by Mass Spectrometry). The PHPBQ was then purified by loading all 100 mL of the incubation mixture on a preparative column (C18, reversed phase column).

Both analytical and preoperative runs were eluted with gradient eluents, running from 0% acetonitrile to 67% acetonitrile (in deionised water) in the presence of 0.1% TFA to keep the pH at 2. During the preparative run, fractions were collected (peaks) and checked for the right product by Mass Spectrometry. Finally, the identified products are dried under vacuum leaving products with ˜99% purity. The ¹H NMR spectrum of the resulting compound is shown in FIG. 2.

PBS=(phosphate buffered saline, pH 7.4)

NaCl, 8.2 g; Na₂₁PO₄.2H₂O, 1.9 g; NaH₂PO₄.H₂O, 0.3 g; Na+, 163, 9 mM; Cl⁻, 140, 3 mM; HPO₄²⁻, 10, 9 mM; H₂PO⁴⁻, 1, 8 mM, Braun Melsungen AG.

### Example 5

### Analysis of Ring-Opened Product

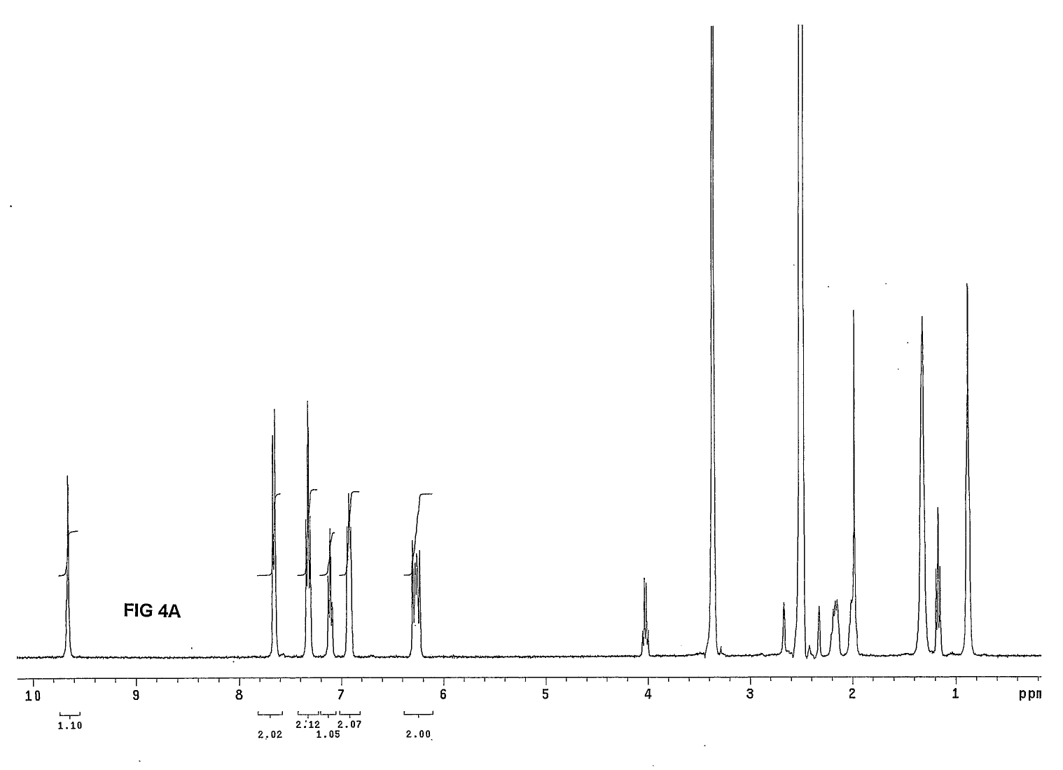

The product of Example 4 (PHBPQ, N-phenyl-2-hydroxy-2 butyl-1,3-propanal quinoneimine) was examined for stability in aqueous solution, followed by analysis by ¹H NMR spectroscopy. FIG. 4A shows the full spectrum and FIG. 4B shows an expansion of the aromatic region.

The results indicated that PHBPQ was more stable at pH 7.4 than 4-OH—OPB with a half-life of ˜2-2.5 hours. The PHBPQ was not re-converted into 4-OH—OPB when the solution was acidified.

### Example 6

### Thiol Derivitisation of Open-Chain Product

To PHBPQ (prepared as described above in Example 4 (500 μM) but remaining in PBS solution) 1.5 mM glutathione was added and incubated for 30 minutes at 37° C. The mixture was purified by the method described above in Example 4. Approximately equal quantities of product derivatised with one glutathione (PHBPQ-1GSH) and two glutathione (PHBPQ-2GSH) were separated from the mixture.

### Example 7

### Suppression of Cytokine Production

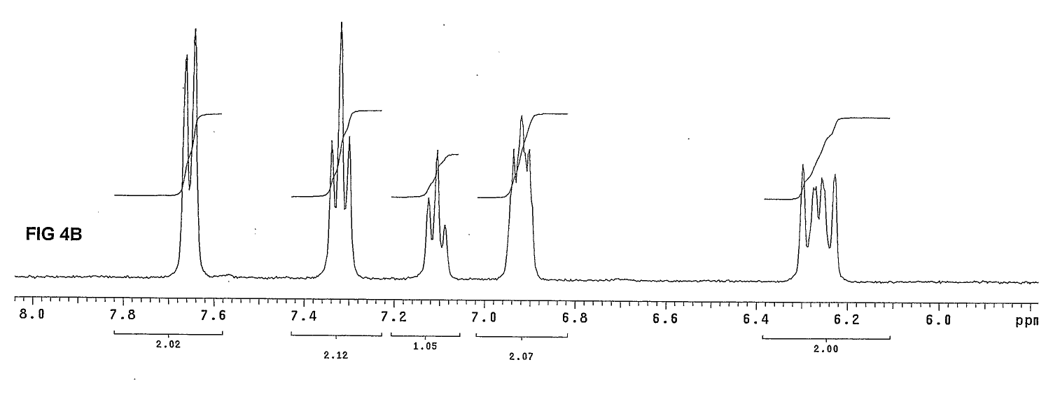

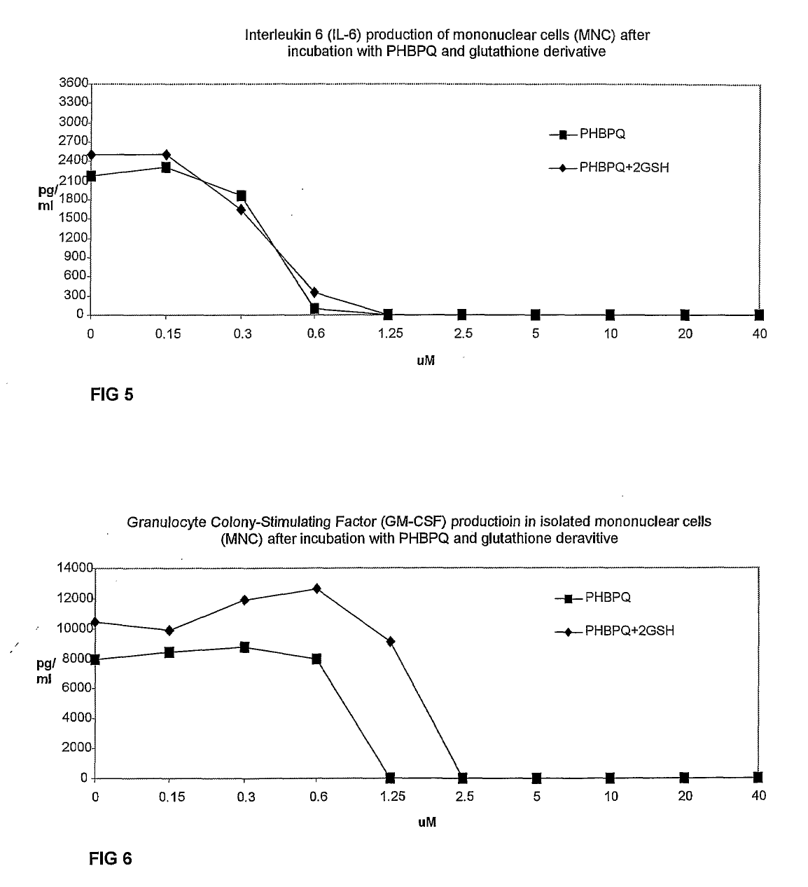

The PHBPQ, as prepared in Example 4 and the PHBPQ-2GSH as prepared in Example 6 were incubated with isolated human mononuclear cells (MNC) derived from peripheral blood from healthy volunteers. Production of the cytokines Interleukin-6 (IL6) and Granulocyte Colony-Stimulating Factor (GM-CSF) was measured. The results (shown in FIGS. 5 and 6) indicates that 0.5-2 FM PHBQ or PHBQ-2GSH is sufficient to completely block production of the measured cytokines.

### Example 8

### Enhancement of Cytokine Production In Vitro

### Method

Human monocytes were grown on a substrate with 10% whole blood taken from healthy volunteers. The monocytes were treated with moderate doses of 4-OH—OPB as prepared in Example 1 (0 to 160 μM) and stimulated with lipooligosaccharide (LOS). The production of interleukins-6 and -8 (IL6 and IL8) and Tumour Necrosis Factor (TNF) and interleukins-1B and -12 by the monocytes was measured.

The experiment was repeated using PHBPQ in place of 4-OH—OPB.

### Results

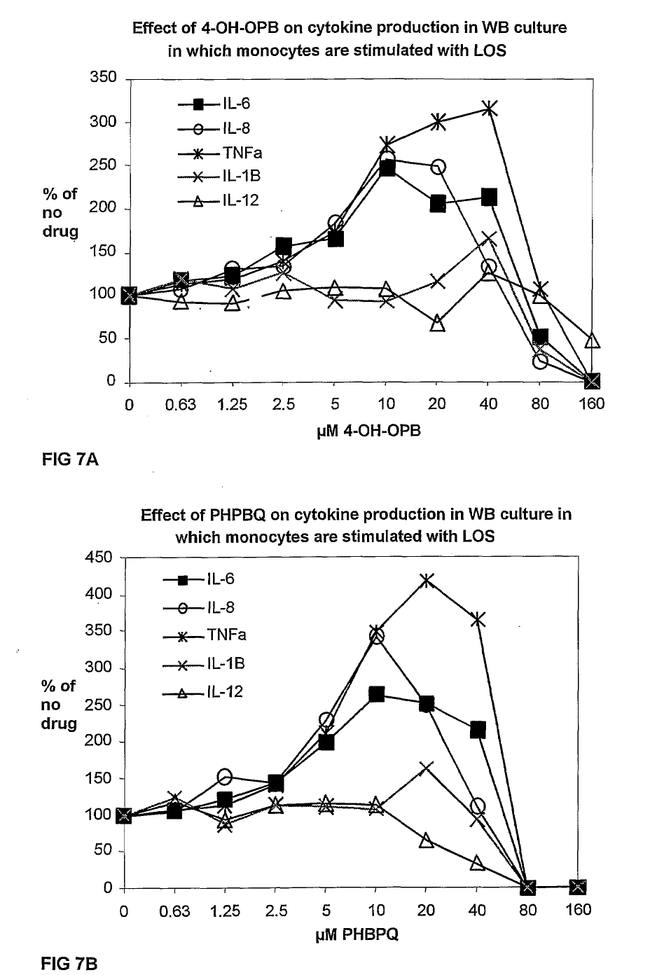

At concentrations up to 40 μM of either compound, increases in the production of all monokines was observed. At concentrations above 40 μM, production of all of these monokines decreased from the peak value and was then suppressed below baseline. The results are shown in FIGS. 7A and 7B

### Lymphocyte Stimulation

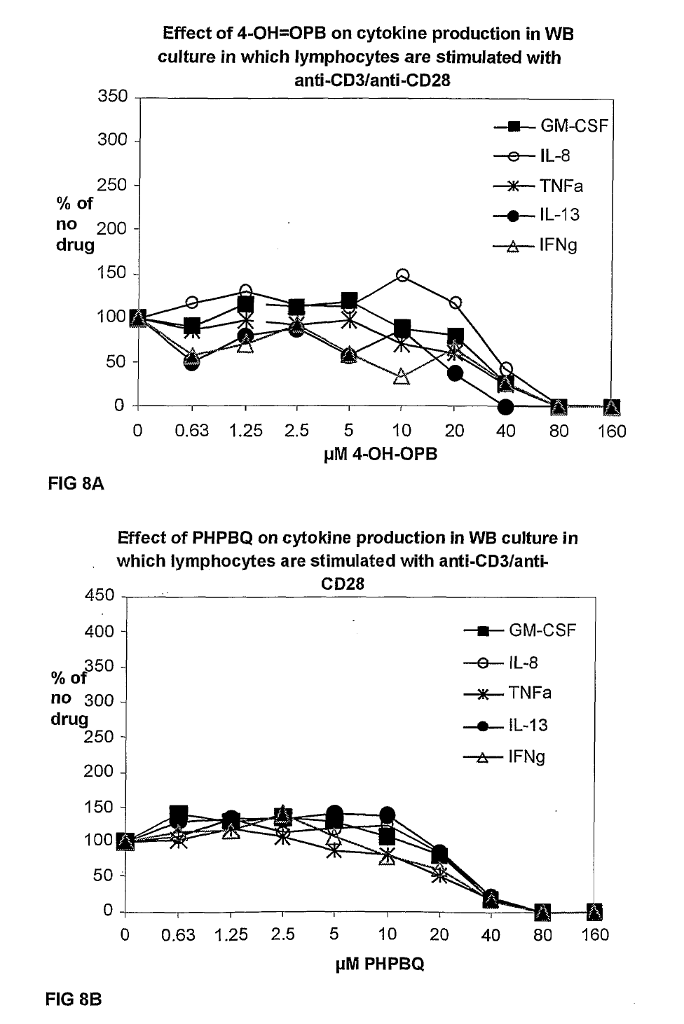

When the above experiment was repeated for lymphocyte stimulation with anti-DC3/anti-CD28 antibodies the resulting increase in expression was less marked but suppression of production was again limited to higher concentrations. The results are shown in FIGS. 8A and 8B.

### Example 9

### Enhancement of Cytokine Production In Vivo and Ex Vivo

Eight of the volunteers received 4-OH—OPB 200 mg orally as two tablets of 100 mg and eight were given placebo tablets. Placebo tablets were chemically identical with the exception of the presence of 4-OH—OPB. The study drug and placebo were administered 2 hours prior to infusion of LPS. LPS (Escheria coli lipopolysaccharide, lot G1, United States Pharmacopeial Convention, Rockville, Md.) was administered as a bolus intravenous injection at a dose of 4 ng/kg body weight.

Blood was drawn directly prior to the administration of 4-OH—OPB, directly prior to injection of LPS and 0.5, 1, 1.5, 2, 3, 4, 5, 6, 8, 10 and 23 hours thereafter for plasma levels of 4-OH—OPB and/or metabolites. Blood was collected in heparinized tubes which were centrifuged at 3000 r.p.m. for 10 minutes at 4° C., after which 2 ml plasma was transferred in acid containing tubes.

Blood for ex vivo production of cytokines in whole blood and mononuclear cell (MNC) cultures was taken directly prior to the administration of 4-OH—OPB and directly prior to the injection of LPS (t=−2 hrs and t=0 hrs). Heparinized whole blood or MNCs were incubated with medium, LOS or anti-CD3/CD28. Supernatant was harvested for cytokine measurement at t=24 and t=72 hrs.

Concentrations of TNF-α, IL-6, IL-8, IL-10 were determined in EDTA-anticoagulated plasma by Cytometric Bead Array (Human BD Cytometric Bead Array Kit, BD Biosciences Pharmingen, San Jose, Calif.).

Injection of the LPS endotoxin induced a febrile response, peaking after 4 hours, together with tachycardia and transient flu-like symptoms, including headache and malaise. No adverse events attributable to 4-OH—OPB were observed. There were no complaints of epigastric pain.

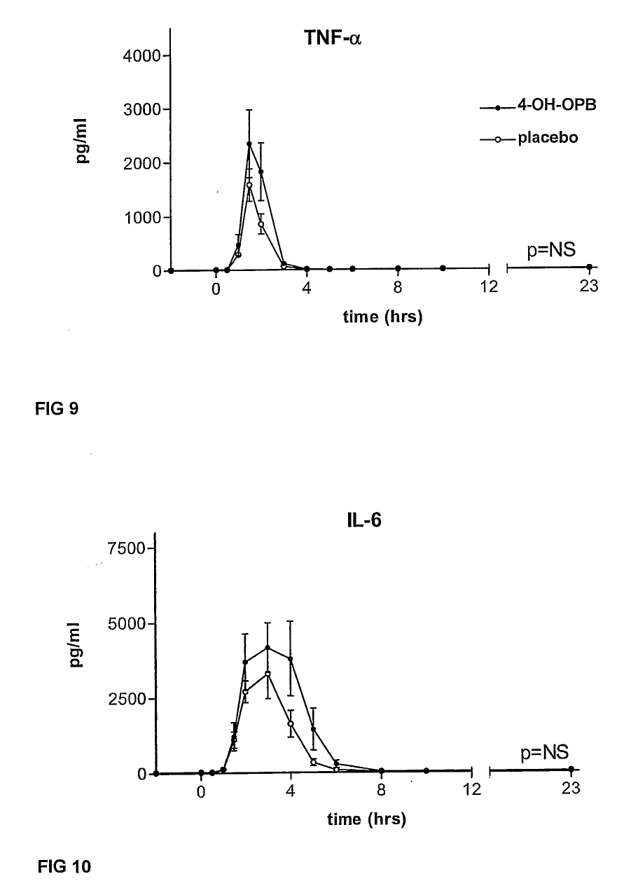

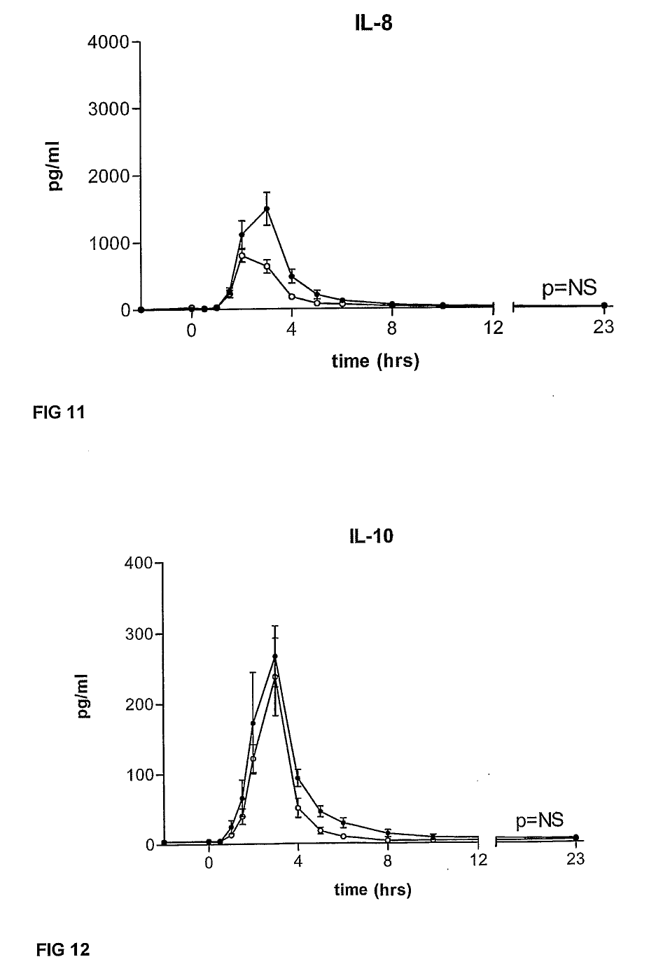

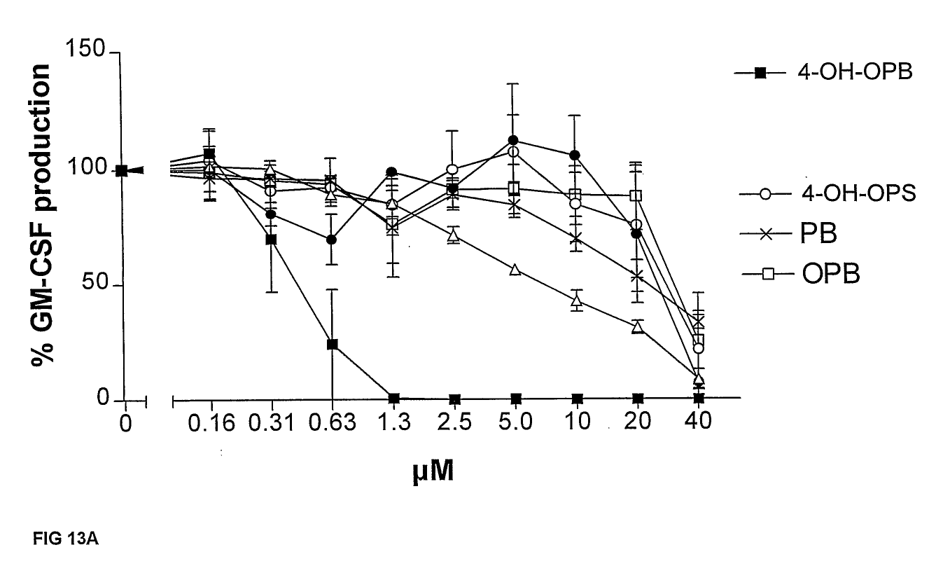

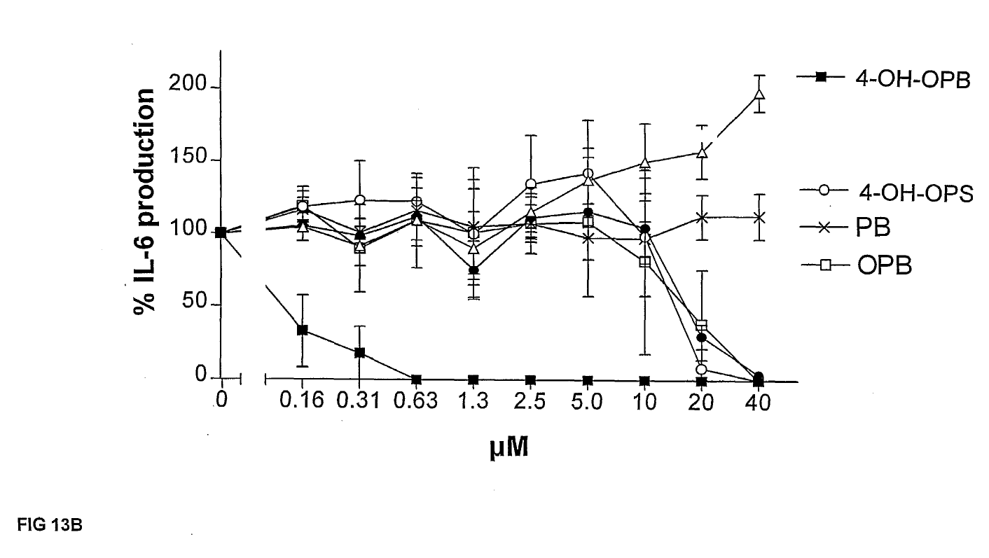

TNF-α plasma levels increase after 1 hour following LPS endotoxin administration, reaching peak values after 1.5 hours. IL-6 en IL-8 levels increased from 90 minutes and peaked after 2.5-3 hrs. Endotoxin also elicited an antiinflammatory cytokine response, as reflected by transient increases in plasma levels of IL-10. The response of these factors with and without the administration of 4-OH—OPB is indicated in FIGS. 9-12. These figures show plasma levels of TNF-α, IL-6, IL-8 and IL-10 respectively in subjects who received an intravenous injection of LPS (4 ng/kg) at t=0 hrs preceded by oral ingestion of placebo (open circles) or 200 mg 4-OH—OPB (closed circles) at t=−2 hrs. Data are mean ±SEM.

### Example 10

### Studies on Further Compounds

### Materials

PB, OPB, 4-OH—OPB and 4-ethylsuphonphenyl-4-hydroxy-1-phenyl-2-hydroxyphenyl-3,5-pyrazoladinedione (referred to as “4-OH—OPS” herein) were tested for effects on various cytokines. 4-OH—OPB and 4-OH—OPS were synthesized by Syntagon (Södertälje, Sweden). PB and OPB were obtained from ICN biomedicals (Aurora, Ohio). 20 mM stocks of the compounds were made in DMSO, which were kept at 4° C.

### Culture Medium

The medium used throughout this Example was Iscoves modified Dulbecco's medium (IMDM, Bio Whittaker, Verviers, Belgium) containing 100 U/ml penicillin, 100 mg/ml streptomycin (Gibco, Merelbeke, Belgium), 50 μM 2-mercaptoethanol (Sigma-Aldrich, Steinheim, Germany), 5% heat-inactivated fetal calf serum (FCS, Bodinco, Alkmaar, the Netherlands) and 20 mg/ml human transferrin (Sigma-Aldrich). All used compounds are endotoxin free.

### Purification and Stimulation of PBMC

PBMC (peripheral blood mononuclear cells) were isolated from freshly drawn blood of a healthy donor and separated over a Percoll gradient (d=1.078, Pharmacia Fine Chemicals AB, Uppsala, Sweden). These isolated PBMC were cultured in 200 μl IMDM (2×105 cells/ml) at 37° C. in the presence of 5% CO₂ in flat bottom plates (Nunc, Roskilde, Denmark). A combination of 1 μg/ml anti-CD3 and anti-CD28 (CLB.T3/4.E res. CLB.CD28/1, CLB, Amsterdam, The Netherlands) was used for T lymphocyte stimulation. For monocyte stimulation 100 pg/ml lipo-oligosaccharide (LOS, kind gift of Dr. J. Poolman, RIVM, Bilthoven, The Netherlands) was used.

### Cytokine Production

Cytokine production of T lymphocytes was measured 3 days after anti CD3/anti CD28 stimulation. 1 day after LOS stimulation of the monocytes, cytokine production was measured. At the indicated times, supernatant was harvested and stored at −20° C. until used. IL-1b, IL-6, IL-8, IL-10, IL-12, IL-13, TNFa and IFNg were detected with ELISA kits (PeliKine-compact, CLB, Amsterdam, The Netherlands) according to the protocol. Summarized briefly, mAbs were coated on flat bottom microtitre plates (Nunc, Maxisorb) overnight in 100 μl 0.1 M sodiumbicarbonate at pH 9.6. All incubations were performed in 100 μl at room temperature. Plates were washed 5 times with PBS+0.02% Tween (Mallinckrodt Baker, Deventer, The Netherlands). Samples were incubated together with a biotinylated mAb for 2 hours in High Performance ELISA-buffer (CLB). Plates were washed 5 times with PBS+0.02% Tween and incubated with streptavidin-labeled poly-horseradish peroxidase (CLB) 1:1000 diluted in PBS+2% skimmed milk for 30 minutes. After 5 wash steps the plates were developed with TMB-substrate containing 0.003% H₂O₂, 100 mg/ml 3,5,3′,5′-tetramethylbenzidine (Merck, Darmstadt, Germany) in 0.11 M sodium acetate, pH 5.5. The reaction was stopped with an equal volume of 2 M H₂SO₄. The plates were measured in an ELISA-reader at 450 nm, using 540 nm for background measurements. GM-CSF was measured using a protocol similar to that used for the other cytokines. The GM-CSF Abs were a kind gift from Dr. G. Trinchieri (the Wistar Institute, Philadelphia, Pa., USA) In this assay the coating Ab was anti-GM-CSF 9.1 (used at 2 μg/ml), the biotinylated Ab was anti-GM-CSF 16.3 (0.1 μg/ml). rGM-CSF (Sandoz, Basel, Switzerland) and HGF 22.10 (CLB) were used for preparation of a standard curve.

### Results:

All five compounds showed cytokine inhibitory activity with 4-hydroxy-oxyphenbutazone as the best inhibitor of IL-6 and GM-CSF production by PBMC in vitro.

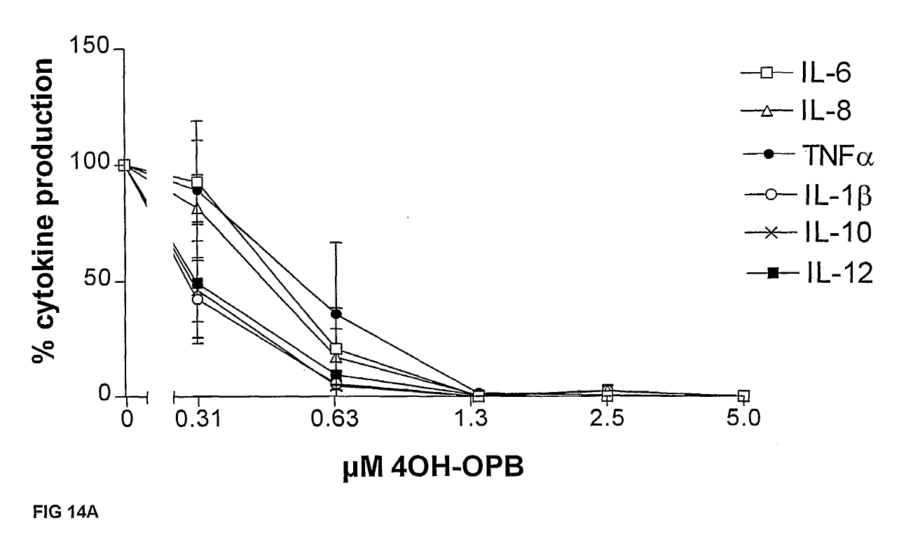

A three days incubation of anti-CD3/anti-CD28 stimulated PBMCs in the presence of a concentration series (up to 40 μM) of the different compounds showed that the GM-CSF production was inhibited impressively when cells were incubated with compound 4-OH—OPB. 1.25 μM was already enough for total inhibition (Table 1, FIG. 13A). 4-OH—OPS and OPB also inhibited the GM-CSF production but at a higher concentration (around 40 μM). PB shows a shallow dose-response curve. Also the LOS induced IL-6 production could be inhibited at a very low concentration of 4-OH—OPB, the inhibiting concentration was even a factor 2 lower (Table 1, FIG. 13B). Again a higher concentration of 4-OH—OPS and OPB was needed compared to 4-OH—OPB.

### Table 1:

IC₅₀ (in μM) of tested compounds of LOS induced IL-6 production and the anti-CD3/anti-CD28 induced GM-CSF production.

TABLE 1
IC₅₀ (in μM) of tested compounds of LOS induced IL-6 production and the
anti-CD3/anti-CD28 induced GM-CSF production.

| Compound | | aCD3/aCD28 |
|---|---|---|
| name/number | LOS stimulation | stimulation |
| PB | >40 | 30 ± 10 |
| OPB | 13 ± 12 | 25 ± 7 |
| 4-OH-OPB | 0.6 ± 0.6 | 0.8 ± 0.5 |
| 4-OH-OPS | 13 ± 4 | 30 ± 10 |

IC₅₀ values (the concentration of the compound needed to inhibit 50% of the maximum response) were determined by using 2-fold titration curves of the compound. The minimum concentration tested was 0.15 μM and the maximum concentration was 40 μM. Results represent the mean ±standard deviation of at least 3 donors. IL-6 in the culture supernatant was determined after 24 h of LOS stimulation of the PBMC by ELISA. GM-CSF in the culture supernatant was determined after 3 days of anti-CD3/anti-CD28 stimulation of the PBMC by ELISA

### Example 11

### Effect of 4-OH—OPB on Other Cytokines

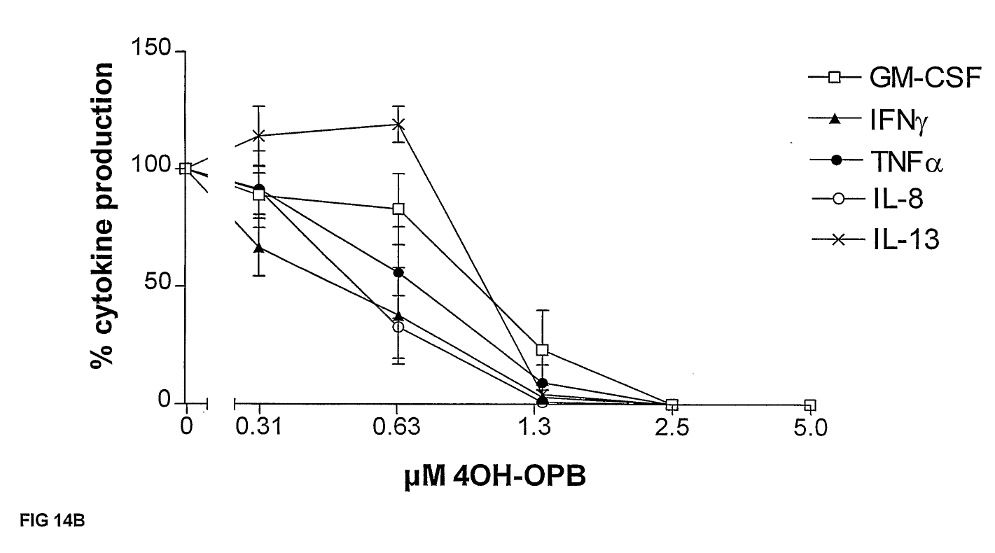

We investigated 4-OH—OPB in more detail. The effect of 4OH—OPB on the LOS-induced IL-6 production was compared with the production of other cytokines produced by the monocytes (FIG. 14A). All monokines were inhibited identically by 4OH—OPB. The inhibition by 4OH—OPB was not stimulus dependent as SAC (heat killed staphylococcus aureus cells) induced monokine production was also inhibited (not shown).

Also the effect of 4OH—OPB on the production of lymphocyte specific cytokines (both Th1 and Th2 specific cytokines) was identical to the inhibition found for GM-CSF (FIG. 14B). Again the inhibition of cytokine production was not stimulus dependent. The cytokine production induced by PMA/ionomycin, PMA/anti-CD28 antibodies or combination of anti-CD2 antibodies/anti-CD28 antibodies was inhibited at similar concentration as seen for the anti-CD3/anti-CD28 stimulation.

1. A method of tonic treatment of an aging mammalian subject, or a subject suffering from mild inflammation, allergy, lupus, fatigue, lethargy or the aftereffects of infection, disease or treatment, comprising administration of a compound of formula I or a salt thereof; 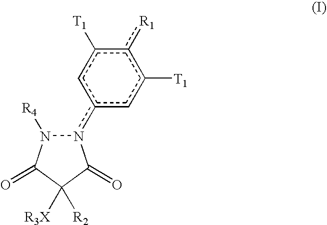
wherein R₁ is H, OH, SH, O-alkyl, S-alkyl, O-acyl or S-acyl, in which case the bond N— —N exists and the six-membered ring is aromatic, or R₁ is O or S joined by a double bond, in which case the bond N— —N is absent and the six-membered ring is 2,5-unsaturated;
R₂ is hydrogen or more preferably an C₁-C₁₀ organic group attached by a carbon atom; X is H, O, OO, S or SS;
R₃ is absent (where X=H), is hydrogen or is a hydroxyl or thiol protecting group;
R₄ is a hetero- or preferably homo-cyclic aryl group, optionally substituted with a further group R₅; and
groups T₁ are each, independently, hydrogen or an S—R₆ group, where each R₆ is independently an organic group of molecular weight up to around 500 amu.

2. A method as claimed in claim 1 wherein the compound of formula I is a compound of formula II; 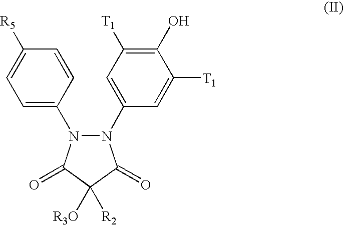
wherein R₅ is an alkyl, alkenyl or alkynyl group (such as those listed infra for R₂), an OH, O-alkyl, O-acyl, SH, S-alkyl, S-acyl, halo, or primary, secondary, tertiary or quaternary amino group;
R₂ is an arylsulphonylalkyl or C₁ to C₆ alkyl, alkenyl, or alkynyl group;
R₃ is hydrogen or a metabolically labile protecting group which yields a physiologically tolerable byproduct;
T₁ is H or a thiol group substituted with an R₆ group (i.e. S—R₆), where R₆ is a targeting moiety or a small (esp MW<500) organic group having at least two functional groups selected from esters, amides, carboxylic acids, hydroxyl groups and amines.

3. A method as claimed in claim 1 wherein the compound of formula I is a compound of formula III; 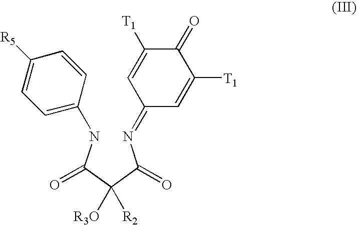
wherein R₅ is an alkyl, alkenyl or alkynyl group (such as those listed infra for R₂), an OH, O-alkyl, O-acyl, SH, S-alkyl, S-acyl, halo, or primary, secondary, tertiary or quaternary amino group;
R₂ is an arylsulphonylalkyl or C₁ to C₆ alkyl, alkenyl, or alkynyl group;
R₃ is hydrogen or a metabolically labile protecting group which yields a physiologically tolerable byproduct;
T₁ is H or a thiol group substituted with an R₆ group (i.e. S—R₆) where R₆ is a targeting moiety or a small (esp MW<500) organic group having at least two functional groups selected from esters, amides, carboxylic acids, hydroxyl groups and amines.

4. A method for the treatment of a mammalian (preferably human) subject comprising administration of a compound of formula I, II or III as defined in claim 1, as a support to another drug and/or treatment regime.

5. A method as claimed in claim 4 wherein, the method is a method for the treatment of a viral, autoimmune, hyperplastic or neoplastic disease.

6. A method as claimed in claim 4 wherein the other drug is an antiviral, an immune suppressant, or an antineoplastic agent and the other treatment regime is surgery and/or external beam irradiation.

7. A method of prophylaxis against the development of diseases associated with old age including arthritis (especially RA), Alzheimer's, Parkinson's and Huntington's diseases, cancer or other neoplastic disease, the method comprising administration of a compound of formula I, II or III as defined in claim 1.

8. A method for the treatment of allergy and/or hypersensitisation comprising administration of a compound of formula I, II or III as defined in claim 1.

9. A method for stimulating cytokine production in a human or non-human animal subject, said method comprising administration of a compound of formula I, II or III as defined in claim 1.

10. A method as claimed in claim 1 wherein said administration is in an amount sufficient to provide a bloodstream concentration of, 0.3 to 160 μM.

11. The use of a compound of formula I, II or III as defined in claim 1 in the manufacture of a medicament for the treatment of the symptoms of inflammation, allergy, fatigue, lethargy or old age or the aftereffects of infection, disease or treatment.

12. A pharmaceutical composition comprising a compound of formula I, II or III as defined in claim 1 and at least one pharmaceutically acceptable excipient, carrier or diluent.

13. A functional or fortified food comprising a compound of formula I, II or III as defined in claim 1 or a salt thereof formulated in an edible food or beverage.

14. A method for the treatment of a mammalian (preferably human) subject comprising administration of a compound of formula I, II or III as defined in claim 2, as a support to another drug and/or treatment regime.

15. A method for the treatment of a mammalian (preferably human) subject comprising administration of a compound of formula I, II or III as defined in claim 3, as a support to another drug and/or treatment regime.

16. A method as claimed in claim 5 wherein the other drug is an antiviral, an immune suppressant, or an antineoplastic agent and the other treatment regime is surgery and/or external beam irradiation.

17. A method of prophylaxis against the development of diseases associated with old age including arthritis (especially RA), Alzheimer's, Parkinson's and Huntington's diseases, cancer or other neoplastic disease, the method comprising administration of a compound of formula I, II or III as defined in claim 2.

18. A method of prophylaxis against the development of diseases associated with old age including arthritis (especially RA), Alzheimer's, Parkinson's and Huntington's diseases, cancer or other neoplastic disease, the method comprising administration of a compound of formula I, II or III as defined in claim 3.

19. A method for the treatment of allergy and/or hypersensitisation comprising administration of a compound of formula I, II or III as defined in claim 2.

20. A method for the treatment of allergy and/or hypersensitisation comprising administration of a compound of formula I, II or III as defined in claim 3.

21. A method for stimulating cytokine production in a human or non-human animal subject, said method comprising administration of a compound of formula I, II or III as defined in claim 2.

22. A method for stimulating cytokine production in a human or non-human animal subject, said method comprising administration of a compound of formula I, II or III as defined in claim 3.

23. A method as claimed in claim 2 wherein said administration is in an amount sufficient to provide a bloodstream concentration of, 0.3 to 160 μM.

24. A method as claimed in claim 3 wherein said administration is in an amount sufficient to provide a bloodstream concentration of, 0.3 to 160 μM.

25. A method as claimed in claim 4 wherein said administration is in an amount sufficient to provide a bloodstream concentration of, 0.3 to 160 μM.

26. A method as claimed in claim 5 wherein said administration is in an amount sufficient to provide a bloodstream concentration of, 0.3 to 160 μM.

27. A method as claimed in claim 6 wherein said administration is in an amount sufficient to provide a bloodstream concentration of, 0.3 to 160 μM.

28. A method as claimed in claim 7 wherein said administration is in an amount sufficient to provide a bloodstream concentration of, 0.3 to 160 μM.

29. A method as claimed in claim 8 wherein said administration is in an amount sufficient to provide a bloodstream concentration of, 0.3 to 160 μM.

30. A method as claimed in claim 9 wherein said administration is in an amount sufficient to provide a bloodstream concentration of, 0.3 to 160 μM.

31. The use of a compound of formula I, II or III as defined in claim 2 in the manufacture of a medicament for the treatment of the symptoms of inflammation, allergy, fatigue, lethargy or old age or the aftereffects of infection, disease or treatment.

32. The use of a compound of formula I, II or III as defined in claim 3 in the manufacture of a medicament for the treatment of the symptoms of inflammation, allergy, fatigue, lethargy or old age or the aftereffects of infection, disease or treatment.

33. A pharmaceutical composition comprising a compound of formula I, II or III as defined in claim 2 and at least one pharmaceutically acceptable excipient, carrier or diluent.

34. A pharmaceutical composition comprising a compound of formula I, II or III as defined in claim 3 and at least one pharmaceutically acceptable excipient, carrier or diluent.

35. A functional or fortified food comprising a compound of formula I, II or III as defined in claim 2 or a salt thereof formulated in an edible food or beverage.

36. A functional or fortified food comprising a compound of formula I, II or III as defined in claim 3 or a salt thereof formulated in an edible food or beverage.
